(19) United States
(12) Patent Application Publication
(10) Pub. No.: US 2003/0195625 A1
(43) Pub. Date: Oct. 16, 2003

(54) Biodegradable follicle hair implant

(76) Inventors: Marco A. Garcia Castro, Mira Mesa, CA (US); Jose L. Garcia Huichan, Mira Mesa, CA (US)

Correspondence Address: MARCO A. GARCIA CASTRO, 568 CANYON DR., BONITA, CA, 91902, US

(21) Appl. No.: 10/121,651

(22) Filed: Apr. 15, 2002

(51) Int. Cl. A61F 2/10

(52) U.S. Cl. 623/15.11; 623/23.75

(57) ABSTRACT

A synthetic or natural human hair end is encapsulated with a biodegradable implantable material, to form an artificial follicle suitable for implantation. The biodegradable material can be programmed to be reabsorbed by the body in a predetermined term of time. Once the material is substantially absorbed by the body the hair shaft will lose its anchor hold into the derma. The degenerated hair will shed by simply combing or washing the hair. The created artificial hair must be implanted individually to create a natural appearance.

### I.) BACKGROUND OF THE INVENTION

1. Field Of The Invention

Is a refilling of my earlier made disclosure No. 480421 filed: Sep. 27, 2000, disclosure No. 508854 filed: Mar. 26, 2002 and prior non-provisional application Ser. No. 09/757090 filed: Jan. 10, 2001 now abandon. This invention relates to an artificial or natural hair filament which terminal is modified and made suitable for implantation into human derma.

2. Description Of The Prior Art

Androgenic alopecia is being treated in many different ways. Scalp lotions, wigs, unguents, medications and hair transplantation. Scalp lotions like Rogaine, will not give back a full head of hair. Hair pieces require costly scheduled maintenance. Medications like Propecia, have side effects. Hair transplantations, are painful, expensive and require successive surgical procedures to accomplish the hair density required for the patient. Besides, hair transplantations require recovery from surgery, it leaves scars and the new hair needs six months to start re-growing.

A recent patent search disclosed two documents which are relevant in light of the instant invention. The first of this is U.S. Pat. No. 5,061284, issued to Laghi in 1991. This patent teaches the coating of a human hair with silicone to form an artificial follicle. The second of this U.S. Pat. No. 4,517,997, issued to Forchetti in 1985. This patent teaches the coating of human hair with gold to form an artificial follicle. The disadvantage of the already mentioned patents is that once the silicone or gold hair follicle is implanted, it will stay inside the derma permanently. Leaving no possibility for the hair to be renewed, unless each of the silicone or gold hair implants is surgically removed from the scalp. One day the hair implanted will wear off and this condition can be deleterious to health by having implanted a prosthetic device that has stop performing its original function. Therefore, one day the implanted follicle made of silicone or gold will have to be removed, leaving the patient with a painful and costly procedure to face in the future. Both patents offer a permanent procedure that needs surgery to be reversed.

There is a necessity for the hair to be replaced periodically because it gradually degenerates through time. This degeneration is called weathering; which is the progressive degeneration of hair from root to tip due to a variety of environmental and cosmetic factors such as: combing, brushing, bleaching, washing, permanent waving, natural friction among hairs, ultra violet radiation and other natural influences. When the hair suffers this cuticle breakdown, it results in a frizzy, coarse, tangling and matting appearance. All these structural abnormalities can be detected by microscopic examination.

The scalp's hair adheres to a genetically programmed scheduled that includes growth, resting and shedding. In general the hair grows one inch per month and continues growing for two to six years. When the growth phase ends, the hair shaft begins a rest period and then sheds. Eventually, a new hair shaft begins to grow from the root embedded inside the hair follicle, pushing out the old shaft as it grows. The distinguishing characteristics between the Laghi patent, the Forchetti patent and the instant inventor's teachings, is that on this invention a biodegradable implantable material is used to form the artificial hair follicle. Such material possessing a preferred CRT/controlled resorption time characteristic.

### II.) SUMMARY OF THE INVENTION

The object of the present invention is to provide an improved hair implant structure suitable for firm implantation directly under the epidermis of human derma. The instant invention consists of a human or artificial hair filament of suitable length on which an artificial follicle is formed. The hair's shaft is coated by an inert implantable material, which extends to a predetermined distance beyond the follicle and protrude outwardly the skin surface. This is done to avoid implant rejection.

The biodegradable material will encapsulate the end of the shaft and part of the inert implantable material. The biodegradable material has two functions: First, its shape will emulate the form of hair follicle in order to provide anchoring between the implanted hair shaft and the derma. Second, to provide a biological clock. By this is meant has follows: the biodegradable material can be programmed to be reabsorbed by the body in a predetermined term of time. Once the material is substantially reabsorbed by the body, it will lose its anchor hold into the derma. The degenerated hair will be released from the skin naturally by combing or washing the hair. As previously mentioned, it is desirable for the scalp to shed every single hair, due to the weathering effect.

The advantages of the present invention are: the hair to be implanted can be from own patient, donor or synthetic, surgical removal of skin strips from hair areas of the patient's scalp is eliminated, recovery period from surgery is eliminated. Biodegradable hair follicles may be single or include more hairs. The implanted hair follicle once degenerated will shed by natural process. The implanted hair is safe, undetectable, and possess natural orientation and look. Furthermore, any level of hair density can be achieved in a single session.

The instant invention avoids the short comings of the latter as well as the former mentioned art by providing a unique HIBC/hair-implant biological clock artificial clock artificial follicle that advantageously will shed naturally. The implantable biodegradable material which forms the hair's follicle can be programmed to be reabsorbed by the body in a predetermined term of time, generally measure in years. Once the biodegradable material is substantially absorbed by the body, the synthetic follicle will automatically lose its anchoring hold into the derma, the degenerated hair will be naturally released by simply combing and washing of the hair. Therefore, the patient will have the option to periodically obtain replacement hairs if desire effectively emulating the natural loss age and replenishment of a normal derma of healthy hair.

### The Biodegradable Material

A resorbable material has the advantage of not having to be removed once it has completed its function. Resorbable sutures, and staples are examples of such materials. There are medical materials available today which have biological, physical and chemical properties necessary for fabrication of devices that can degrade inside the body without detrimental consequences. Polymers such as: polylactides, polyglicolides, polyanhydrides and polyorthoesters. Colagen, hyaluronic acid, and glycosaminoglycans are examples of implantable materials which resorb. The rates of resorption are limited to the nature of the particular material but modifications can be made to change the rate of resorption. An implantable device should have mechanical properties such as: tensile strength, formability, elasticity, controlled resorption and be physiologically acceptable.

Copolymers and silk homopolymers have found use in the production of abundant implantable devices. These implantable devices are made of recombinant chains of copolymers with alternative segments of repetitive units. It is well known by those related to the art that by varying the length, ratio, molecular weight and by sequence modification the rate of resorption can be greatly modified. The method of preparation of these copolymers can be made by preparing in advance a synthetic DNA sequence, the monomers of which will clone to ensure integrity of said sequence, as for example:

Sequence description: ID NO:X

```
Val Pro Gly Val Gly Val Pro Gly  Val Gly Val
1 5 10 15
Pro Gly Val Gly Val Pro Gly Val Gly Val Pro Gly Val Gly Val Pro
                        20 25 30
Gly Val Gly Val Pro Gly Val Gly Gly Ala Gly Ala Gly Ser Gly Ala
                35 40 45
Gly Ala Gly Ser Val Pro Gly Val Gly Val Pro Gly Val Gly Val Pro
        50 55 60
Gly Val Gly Val Pro Gly Val Gly Val Pro Gly Val Gly Val Pro Gly
65 70 75 80
Val Gly Val Pro Gly Val Gly Val Pro Gly Val Gly Gly Ala Gly Ala
                                85 90 95
Gly Ser Gly Ala Gly Ala Gly Ser Val Pro Gly Val Gly Val Pro Gly
                          100 105 110
Gly Val Gly Val Pro Gly Val Gly Val Pro Gly Val Gly Val Pro Gly
                275 280 285
Val Gly Val Pro Gly Val Gly Val Pro Gly Val Gly Gly Ala Gly Ala
        290 295 300
Gly Ser Gly Ala Gly Ala Gly Ser Val Pro Gly Val Gly Val Pro Gly
305 310 315 320
Val Gly Val Pro Gly Val Gly Val Pro Gly Val Gly Val Pro Gly Val
                                325 330 355
Gly Val Pro Gly Val Gly Val Pro Gly Val Gly Val Pro Gly Val Gly
                        340 345 350
Gly Ala Gly Ala Gly Ser Gly Ala Gly Ala Gly Ser Val Pro Gly Val
                355 360 365
Gly Val Pro Gly Val Gly Val Pro Gly Val Gly Val Pro Gly Val Gly
        370 375 380
Val Pro Gly Val Gly Val Pro Gly Val Gly Val Pro Gly Val Gly Val
385 390 395 400
Pro Gly Val Gly Gly Ala Gly Ala Gly Ser Gly Ala Gly Ala Gly Ser
                                405 410 415
Val Pro Gly Val Gly Val Pro Gly Val Gly Val Pro Gly Val Gly Val
                        420 425 430
Pro Gly Val Gly Val Pro Gly Val Gly Val Pro Gly Val Gly Val Pro
                435 440 445
Gly Val Gly Val Pro Gly Val Gly Gly Ala Gly Ala Gly Ser Gly Ala
        450 455 460
Gly Ala Gly Ser Val Pro Gly Val Gly Val Pro Gly Val Gly Val Pro
465 470 475 480
Val Gly Val Pro Gly Val Gly Val Pro Gly Val Gly Gly Ala Gly Ala
                        500 505 510
Gly Ser Gly Ala Gly Ala Gly Ser Val Pro Gly Val Gly Val Pro Gly
                515 520 525
Val Gly Val Pro Gly Val Gly Val Pro Gly Val Gly Val Pro Gly Val
        530 535 540
Gly Val Pro Gly Val Gly Val Pro Gly Val Gly Val Pro Gly Val Gly
545 550 555 560
Gly Ala Gly Ala Gly Ser Gly Ala Gly Ala Gly Ser Val Pro Gly Val
                                565 570 575
Gly Val Pro Gly Val G ly Val Pro Gly Val Gly Val Pro Gly Val Gly
                        580 585 590
Val Pro Gly Val Gly Val Pro Gly Val Gly Val Pro Gly Val Gly Val
                595 600 605
Pro Gly Val Gly Gly Ala Gly Ala Gly Ser Gly Ala Gly Ala Gly Ser
        610 615 620
Val Pro Gly Val Gly Val Pro Gly Val Gly Val Pro Gly Val Gly Val
625 630 635 640
Pro Gly Val Gly Val Pro Gly Val Gly Val Pro Gly Val Gly Val Pro
                                645 650 655
Gly Val Gly Val Pro Gly Val Gly Gly Ala Gly Ala Gly Ser Gly Ala
                        660 665 670
Gly Ala Gly Ser Val Pro Gly Val Gly Val Pro Gly Val Gly Val Pro
                675 680 685
Gly Val Gly Val Pro Gly Val Gly Val Pro Gly Val Gly Val Pro Gly
        690 695 700
Val Gly Val Pro Gly Val Gly Val Pro Gly Val Gly Gly Ala Gly Ala
705 710 715 720
Gly Ser Gly Ala Gly Ala Gly Ser Val Pro Gly Val Gly Val Pro Gly
                                725 730 735
Val Gly Val Pro Gly Val Gly Val Pro Gly Val Gly Val Pro Gly Val
                        740 745 750
Gly Val Pro Gly Val Gly Val Pro Gly Val Gly Val Pro Gly Val Gly
                755 760 765
Gly Ala Gly Ala Gly Ser Gly Ala Gly Ala Gly Ser Val Pro Gly Val
        770 775 780
Gly Val Pro Gly Val Gly Val Pro Gly Val Gly Val Pro Gly Val Gly
785 790 795 800
Val Pro Gly Val Gly Val Pro Gly Val Gly Val Pro Gly Val Gly Val
                                805 810 815
Pro Gly Val Gly Gly Ala Gly Ala Gly Ser Gly Ala Gly Ala Gly Ser
                        820 825 830
Val Pro Gly Val Gly Val Pro Gly Val Gly Val Pro Gly Val Gly Val
                835 840 845
Pro Gly Val Gly Val Pro Gly Val Gly Val Pro Gly Val Gly Val Pro
        850 855 860
Gly Val Gly Val Pro Gly Val Gly Gly Ala Gly Ala Gly Ser Gly Ala
865 870 875 880
Gly Ala Gly Ser Val Pro Gly Val Gly Val Pro Gly Val Gly Val Pro
                                885 890 895
Gly Val Gly Val Pro Gly Val Gly Val Pro Gly Val Gly Val Pro Gly
                        900 905 910
Val Gly Val Pro Gly Val Gly Val Pro Gly Val Gly Gly Ala Gly Ala
                915 920 925
Gly Ser Gly Ala Gly Ala Gly Ser
        930 935
(1) SEQUENCE CHARACTERISTIC:
        (A) LENGTH: 936 amino acids
        (B) TYPE: amino acid
        (C) STRANDEDNESS: single
        (D) TOPOLOGY: linear
```

### III.) BRIEF DESCRIPTION OF THE DRAWINGS

FIG. 1 is an illustration of a single strand embodiment of the invention;

FIG. 2 is an illustration, in partial cross section showing the single strand embodiment of the invention.

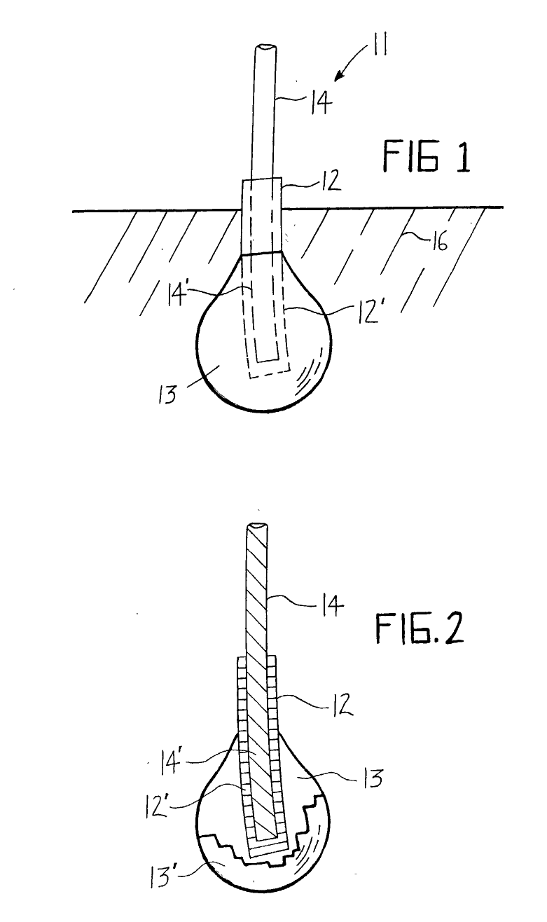

What is claim is:

1.) A method for preparing human or synthetic hair grafts for implantation into human derma, said method comprising: human or synthetic strands of suitable length, with a hair shaft base end and a distal end. The coating of said hair shaft base end and part of the distal end by an implantable inert material. Said hair graft extending a predetermined distance beyond the follicle and protruding outwardly of the skin surface.

2.) The method of claim 1 wherein the coating of said shaft base end and part of the distal end is made by an implantable inert material.

3.) The invention of claim-2, wherein the encapsulation of the hair shaft base end and part of the inert material by a biodegradable implantable material, said encapsulation emulating the shape of a hair follicle. Said material can be a natural or synthetic biodegradable polymer, a biodegradable silicon, or any biodegradable implantable material that is free of immunogenicity, that has mechanical properties that match the application, and that can be programmed for a timed-bioerosion.

4.) The invention of claim-3, wherein the material emulating the follicle in this example is a bioresorbable polypeptide copolymer, wherein said preparation of the material emulating the hair follicle can be made by following a synthetic DNA sequence, wherein said material emulating the follicle is implantable, wherein said material emulating the follicle is sterilized.

5.) The invention of claim-4, wherein said material forming the follicle posses a “CRT” to ensure the mechanical hold into the derma during the predetermined time required for structure integrity, wherein said “CRT” being controlled by varying the length of repetitive units of copolymers.

6.) The invention of claim-5, wherein said material forming the follicle is a “HIBC” structure for implantation below the skin surface, said structure comprising: an implantable synthetic or human hair filament having a distal end and a hair shaft base end, Wherein bearing on said shaft base end is a bioresorbable polypeptide copolymer, said copolymer emulating the form of a follicle and encapsulating both said hair shaft base end and part of said coating of said inert material.

7.) The invention of claim-6, wherein said hair-implant biological clock structure is pre-cured before implantation, wherein said hair-implant can be implanted downwardly into the subcutaneous level.

8.) The invention of claim-7, wherein the biodegradable material is substantially absorbed by the body in a predetermined term of time, generally measured in years.

9.) The invention of claim-8, wherein the degenerated hair shaft will be naturally released from the scalp by the every day process of combing or washing of the hair.
